(19) United States
(12) Patent Application Publication
(10) Pub. No.: US 2016/0074428 A1
(43) Pub. Date: Mar. 17, 2016

(54) COMPOSITIONS AND METHODS FOR THE TREATMENT OF HUMAN IMMUNODEFICIENCY VIRUS INFECTION

(71) Applicant: THE CHILDREN'S HOSPITAL OF PHILADELPHIA, Philadelphia, PA (US)

(72) Inventors: Steven D. Douglas, Philadelphia, PA (US); Florin Tuluc, Cheltenham, PA (US); Sergurei Spitsin, Cherry Hill, NJ (US); John Meshki, Philadelphia, PA (US)

(73) Assignee: THE CHILDREN'S HOSPITAL OF PHILADELPHIA, Philadelphia, PA (US)

(21) Appl. No.: 14/888,549

(22) PCT Filed: May 2, 2014
(86) PCT No.: PCT/US14/36672

§ 371 Date: Nov. 2, 2015

Related U.S. Application Data
(60) Provisional application No. 61/818,688, filed on May 2, 2013.

(51) Int. Cl. A61K 31/713; G01N 33/569

(52) U.S. Cl. CPC A61K 31/713; G01N 33/56972; G01N 2333/70596; G01N 2500/04; G01N 2500/10

(57) ABSTRACT

In accordance with the present invention, a method for inhibiting HIV infection and/or latency in a patient in need thereof is provided. An exemplary method entails administration of an effective amount of an agent which inhibits CD163 receptor activity in a target cells, wherein inhibition of CD163 activity is effective to impede HIV uptake into target monocytes. In another aspect of the invention, a screening method for identifying agents useful for the treatment of HIV infection is disclosed. An exemplary method entails incubating a population of monocytes expressing CD163 in the presence and absence of said test agent, b) contacting said monocytes with a compound that detectably labels said CD163, thereby determining CD163 expression levels in the presence or absence of said agent, agents which inhibit expression of CD163 in treated cells relative to untreated cells being useful for inhibiting HIV infection in said cell.

This application claims priority to U.S. Provisional Application No. 61/818,688 filed May 2, 2013, the entire contents being incorporated herein by reference as though set forth in full.

Pursuant to 35 U.S.C. §202(c) it is acknowledged that the U.S. Government has rights in the invention described herein, which was made with funds from the National Institutes of Health, Grant Numbers RO1-MH49981 and P30 AI45008.

### FIELD OF THE INVENTION

This invention relates to the fields of signal transduction and viral replication. More specifically, the invention provides compositions and methods useful for inhibiting viral replication and latency in HIV infected individuals.

### BACKGROUND OF THE INVENTION

Several publications and patent documents are cited throughout the specification in order to describe the state of the art to which this invention pertains. Each of these citations is incorporated herein by reference as though set forth in full.

Substance P (SP) is an undecapeptide that functions as a neurotransmitter in the central and peripheral nervous system and has immunomodulatory properties. SP belongs to the tachykinin family of neuropeptides, which share a common C-terminal sequence, Phe-X-Gly-Leu-Met-NH2 that is essential for interaction with their receptors (1-3).

Two isoforms of NK1R are generated through alternative splicing: a full length NK1R that includes in its primary structure 407 amino acid residues and a truncated NK1R that lacks the last 96 amino acid residues at the C-terminus intracellular domain (7-9).

Our group has provided compelling evidence that SP and neurokinin-1 receptor (NK1R) are important in pathogenesis of HIV/AIDS (10-13). Plasma SP levels are elevated in HIV-infected individuals (10,14,15) and SP treatment of MDM promotes HIV infection, whereas treatment with NK1R antagonists inhibits HIV infection (12,13,16). However, the molecular mechanisms responsible for the effect of SP of promoting HIV replication in macrophages are not completely understood.

CD163, the scavenger receptor for hemoglobin-haptoglobin complexes, has been linked to HIV infection (17-22). CD163 is expressed exclusively in cells of the monocyte/macrophage lineage (23-25). Hemolysis is caused by infectious and inflammatory conditions and the clearance of hemoglobin-haptoglobin complexes by CD163-linked mechanism is an important process in the resolution of inflammation (26). CD163 expression is upregulated by anti-inflammatory mediators such as glucocorticoids (27) and interleukin-10 (IL-10) (28), and it is down regulated by pro-inflammatory molecules such as IFN-γ, TNF-α and LPS (28). Higher levels of CD163 are detected on macrophages with anti-inflammatory potential such as alternatively activated and deactivated macrophages.

High expression of CD163 was detected on CNS macrophages in brains of HIV positive individuals (29-32). Increased levels of CD163 may be a potential biomarker reflecting efforts of the immune system to resolve immune activation and inflammation in HIV-infected individuals (18). Higher frequency of CD163+/CD16+ cells in HIV positive individuals is associated with a decrease in CD4+ T cells and increase in viral loads (29,32). CD163+/CD16+ macrophages are associated with CNS and other tissue invasion in humans and in non-human primates (29,32). Pro-inflammatory stimuli not only inhibit production of CD163 but also result in shedding of the extracellular portion of CD163 which circulates in blood as a soluble protein (sCD163). Although the functions of sCD163 are unknown, increased levels of sCD163 occur in several chronic inflammatory diseases including HIV infection (17,20,33). Monocyte- and macrophage-derived sCD163 may be a marker of HIV activity that links viral replication with monocyte and macrophage activation (17,20). Inverse correlation was found between membrane CD163 expression in human monocytes and sCD163 levels in plasma (39) suggesting that plasma sCD163 is derived from circulating monocytes and tissue macrophages. There is no direct evidence, however, that soluble or membrane bound CD163 influences HIV entry or replication.

### SUMMARY OF THE INVENTION

In accordance with the present invention, a method for inhibiting HIV infection and/or latency in a patient in need thereof is provided. An exemplary method entails administration of an effective amount of an agent which inhibits CD163 receptor activity in a target cells, wherein inhibition of CD163 activity is effective to impede HIV uptake into target monocytes.

In another aspect of the invention, a screening method for identifying agents useful for the treatment of HIV infection is disclosed. An exemplary method entails incubating a population of monocytes expressing CD163 in the presence and absence of said test agent, b) contacting said monocytes with a compound that detectably labels said CD163, thereby determining CD163 expression levels in the presence or absence of said agent, agents which inhibit expression of CD163 in treated cells relative to untreated cells being useful for inhibiting HIV infection in said cell.

### BRIEF DESCRIPTION OF THE DRAWINGS

FIG. 1. SP-induced intracellular calcium increase in human monocytes is mediated by NK1R. Freshly isolated monocytes were loaded with Fluo-4 AM and Fura-red AM, and used for intracellular calcium measurements by flow cytometry. Pseudo color dot plots and median fluorescence intensities line graphs are shown. a) Forward and side-scatter plots of monocytes. b,c,d) Monocytes were stimuated with 1, 3 or 10 μM SP. e) Monocytes were stimuated with 100 nM fMLP. f) Monocytes were pretreated with 50 μM Aprepitant for 15 minutes followed by 10 μM SP. g,h) Monocytes were treated with either 10 μM neurokinin A (NKA) or 10 μM neurokinin B (NKB), SP was added to the experiments to demonstrate that cells were responsive.

FIG. 2. SP and IL-10 increases expression of CD163 is human macrophages. Freshly isolated monocytes were treated with SP or IL-10, then stained for CD163 and analyzed by flow cytometry. a) Time course of monocytes treated with 10 μM SP for 2, 4 or 6 days. b) Dose response of monocytes cultured for 4 days in the presence of 0, 0.3, 1, 3, 10 or 30 μM SP. c) Time course of monocytes treated with 20 ng/ml IL-10 for 2, 4 or 6 days. d) Dose response of monocytes cultured for 4 days in the presence of 0.06, 0.2, 0.6, 2, 6 or 20 ng/ml IL-10.

FIG. 3. Monocytes expressing high levels of CD163 are more susceptible to HIV infection. a) Monocytes were sorted based on expression level of CD163 using a MoFlo XDP (Beckman Coulter) cell sorter and infected with HIV-Bal 4 hours later. Cell purity was checked after each cell sorting experiment and found higher than 95%. b) HIV GAG normalized to GAPDH assayed by real time RT PCR. Average results of three independent experiments are presented as mean±SD. * p<0.01 CD163^{high} versus CD163^{low} cultures by Student's t test.

FIG. 4. Decreased expression of CD163 on macrophages leads to inhibition of HIV infection. Macrophages were cultured in vitro and siRNA was used to decrease the expression of CD163. a) Flow cytometry data of CD163 expression in macrophages tranfected with either CD163 siRNA or control siRNA for 48 hours. b) Macrophages, transfected with CD163 siRNA, control siRNA or untransfected control, were infected after 48 hours with HIV-BaL, RNA was collected at indicated time points and assayed for HIV GAG by real time RT PCR. Average results of three independent experiments are presented as mean±SD. * p<0.01 anti-CD163 siRNA versus CD163 siRNA samples by Student's t test.

FIG. 5. Effect of HbHp-complexes on HIV infection in macrophages. Hb and Hp were added to macrophages in a 1:1 molar ratio and incubated for 2 h at 37° C. a) Macrophages, treated with 0, 2, 10 or 20 μg/ml of HbHp complex and infected with HIV-BaL, were assayed 7 days postinfection for HIV GAG by real time RT PCR. b) Macrophages, treated with 0, 2, 10 or 50 μg/ml of HbHp complex and infected with HIV-GFP-tagged reported virus, were assayed 7 days postinfection for GFP fluorescence. c) Representative images of macrophages, treated with 0, 2, 10, and 50 μg/ml of HbHp complex and infected with HIV-GFP-tagged reported virus 7 days postinfection. Average results of three independent experiments are presented as mean±SD.

### DETAILED DESCRIPTION OF THE INVENTION

Our study demonstrates that SP induces NK1R-mediated intracellular calcium increase in freshly-isolated human monocytes and triggers the alternative pathway of macrophage differentiation, with upregulation of membrane bound CD163. We also demonstrate that the productivity of infection is increased in macrophages with high levels of expression of CD163. Our findings provide novel insights into the cellular mechanisms responsible for SP enhancement of HIV infection.

SP enhanced CD163 expression on monocytes in a dose- and time-dependent manner. We found that the productivity of HIV infection was higher in CD163^{high} cells. Additionally, in macrophages with CD163 expression knocked down, we found a significant decrease of HIV infection. The expression of CD163 on macrophages, in addition to being a prognostic marker of HIV infection, may also be critical in HIV immunopathogenesis.

### I. Definitions

Various terms relating to the biological molecules of the present invention are used hereinabove and also throughout the specifications and claims. The terms “specifically hybridizing,” “percent similarity” and “percent identity (identical)” are defined in detail in the description set forth below.

With reference to nucleic acids of the invention, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote.

With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

With respect to protein, the term “isolated protein” or “isolated and purified protein” is sometimes used herein. This term refers primarily to a protein produced by expression of an isolated nucleic acid molecule of the invention. Alternatively, this term may refer to a protein which has been sufficiently separated from other proteins with which it would naturally be associated, so as to exist in “substantially pure” form.

The phrase “consisting essentially of” when referring to a particular nucleotide or amino acid means a sequence having the properties of a given SEQ ID No:. For example, when used in reference to an amino acid sequence, the phrase includes the sequence per se and molecular modifications that would not affect the basic and novel characteristics of the sequence.

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, and the like).

With respect to antibodies of the invention, the term “immunologically specific” refers to antibodies that bind to one or more epitopes of a protein of interest (e.g., CD163), but which do not substantially recognize and bind other molecules in a sample containing a mixed population of antigenic biological molecules.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunloglobulin molecule such as those portions known in the art as Fab, Fab′, F(ab′)2 and F(v).

With respect to oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The present invention also includes active portions, fragments, derivatives and functional mimetics of the CD163 polypeptide or protein of the invention.

An “active portion” of CD163 polypeptide means a peptide which is less than said full length CD163 polypeptide, but which retains its essential biological activity, e.g., transcription regulating activity.

A “fragment” of the CD163 polypeptide means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids. Fragments of the CD163 polypeptide sequence, antigenic determinants or epitopes are useful for raising antibodies to a portion of the CD163 amino acid sequence. Certain fragments may be useful to block CD163 receptor action.

A “derivative” of the CD163 polypeptide or a fragment thereof means a polypeptide modified by varying the amino acid sequence of the protein, e.g. by manipulation of the nucleic acid encoding the protein or by altering the protein itself. Such derivatives of the natural amino acid sequence may involve insertion, addition, deletion or substitution of one or more amino acids, without fundamentally altering the essential activity of the wildtype CD163 polypeptide.

“Functional mimetic” means a substance which may not contain an active portion of the CD163 amino acid sequence, and probably is not a peptide at all, but which retains the essential biological activity of natural CD163 polypeptide.

A “promoter” is a DNA sequence located proximal to the start of transcription at the 5′ end of an operably linked transcribed sequence. The promoter may contain one or more regulatory elements or modules which interact in modulating transcription of the operably linked gene.

“Operably linked” describes two macromolecular elements arranged such that modulating the activity of the first element induces an effect on the second element. In this manner, modulation of the activity of a promoter element may be used to alter and/or regulate the expression of an operably-linked coding sequence. For example, the transcription of a coding sequence that is operably-linked to a promoter element is induced by factors that “activate” the promoter's activity; transcription of a coding sequence that is operably-linked to a promoter element is inhibited by factors that “repress” the promoter's activity. Thus, a promoter region is operably-linked to the coding sequence of a protein if transcription of such coding sequence activity is influenced by the activity of the promoter.

As used herein, an “expression vector” is a vector especially designed to provide an environment which allows the expression of the cloned gene after transformation into the host. One manner of providing such an environment is to include transcriptional and translational regulatory sequences on such expression vehicle, such transcriptional and translational regulatory sequences being capable of being operably linked to the cloned gene. Another manner of providing such an environment is to provide a cloning site or sites on such vehicle, wherein a desired cloned gene and desired expression regulatory elements may be cloned.

In an expression vector, the gene to be cloned is usually operably-linked to certain control sequences such as promoter sequences. Expression control sequences will vary depending on whether the vector is designed to express the operably-linked gene in a prokaryotic or eukaryotic host and may additionally contain transcriptional elements such as enhancer elements, termination sequences, tissue-specificity elements, and/or translational initiation and termination sites.

Sequence information for CD163 is available on GenBank.

### II. CD163 Protein and Antibodies

Purified CD163, or fragments thereof, may be used to produce polyclonal or monoclonal antibodies which also may serve as sensitive detection reagents for the presence and accumulation of CD163 (or complexes containing CD163) in mammalian cells. Recombinant techniques enable expression of fusion proteins containing part or all of the CD163 protein. The full length protein or fragments of the protein may be used to advantage to generate an array of monoclonal antibodies specific for various epitopes of the protein, thereby providing even greater sensitivity for detection of the protein in cells.

Polyclonal or monoclonal antibodies immunologically specific for CD163 may be used in a variety of assays designed to detect and quantitate the protein. Such assays include, but are not limited to: (1) flow cytometric analysis; (2) immunochemical localization of CD163 in brain cells and other cells infected with HIV; and (3) immunoblot analysis (e.g., dot blot, Western blot) of extracts from various cells. Additionally, as described above, anti-CD163 can be used for purification of CD163 (e.g., affinity column purification, immunoprecipitation).

From the foregoing discussion, it can be seen that CD163-encoding nucleic acids, CD163 expressing vectors, CD163 proteins and anti-CD163 antibodies of the invention can be used to detect CD163 gene expression in HIV infected to cells and alter CD163 protein accumulation for purposes of assessing the genetic and protein interactions involved in neurodestructive diseases such as HIV.

Exemplary approaches for detecting CD163 nucleic acid or polypeptides/proteins include: a) comparing the sequence of nucleic acid in the sample with the CD163 nucleic acid sequence to determine whether the sample from the patient contains mutations; or b) determining the presence, in a sample from a patient, of the polypeptide encoded by the CD163 gene and, if present, determining whether the polypeptide is full length, and/or is mutated, and/or is expressed at the normal level; or c) using DNA restriction mapping to compare the restriction pattern produced when a restriction enzyme cuts a sample of nucleic acid from the patient with the restriction pattern obtained from normal CD163 gene or from known mutations thereof; or, d) using a specific binding member capable of binding to a CD163 nucleic acid sequence (either normal sequence or known mutated sequence), the specific binding member comprising nucleic acid hybridizable with the CD163 sequence, or substances comprising an antibody domain with specificity for a native or mutated CD163 nucleic acid sequence or the polypeptide encoded by it, the specific binding member being labeled so that binding of the specific binding member to its binding partner is detectable; or, e) using PCR involving one or more primers based on normal or mutated CD163 gene sequence to screen for normal or mutant CD163 gene in a sample from a patient.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples and they do not need to be listed here. Further, the term “specific binding pair” is also applicable where either or both of the specific binding member and the binding partner comprise a part of a large molecule. In embodiments in which the specific binding pair are nucleic acid sequences, they will be of a length to hybridize to each other under conditions of the assay, preferably greater than 10 nucleotides long, more preferably greater than 15 or 20 nucleotides long.

The identification of the CD163 gene and its association with infectious disease and neurodegenerative disorders paves the way for aspects of the present invention to provide the use of materials and methods, such as are disclosed and discussed above, for establishing the contribution of this protein to the control of gene expression during HIV-1 induced brain injury.

Such knowledge should facilitate planning of appropriate therapeutic and/or prophylactic measures, permitting stream-lining of treatment. The approach further stream-lines treatment by targeting those patients most likely to benefit.

According to another aspect of the invention, methods of screening agents to identify suitable drugs for inhibiting CD163 function are provided.

The CD163 polypeptide or fragment employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between a CD163 polypeptide or fragment and the agent being tested, or examine the degree to which the formation of a complex between a CD163 polypeptide or fragment and a known ligand is interfered with by the agent being tested.

Another technique for drug screening provides high throughput screening for compounds having suitable binding affinity to the CD163 polypeptides and is described in detail in Geysen, PCT published application WO 84/03564, published on Sep. 13, 1984. Briefly stated, large numbers of different, small peptide test compounds are synthesized on a solid substrate, such as plastic pins or some other surface. The peptide test compounds are reacted with CD163 polypeptide and washed. Bound CD163 polypeptide is then detected by methods well known in the art.

Purified CD163 can be coated directly onto plates for use in the aforementioned drug screening techniques. However, non-neutralizing antibodies to the polypeptide can be used to capture antibodies to immobilize the CD163 polypeptide on the solid phase.

This invention also contemplates the use of competitive drug screening assays in which neutralizing antibodies capable of specifically binding the CD163 polypeptide compete with a test compound for binding to the CD163 polypeptide or fragments thereof. In this manner, the antibodies can be used to detect the presence of any peptide which shares one or more antigenic determinants of the CD163 polypeptide.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, one first determines the three-dimensional structure of a protein of interest (e.g., CD163 polypeptide) or, for example, of the CD163-DNA complex, by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides (e.g., CD163 polypeptide) may be analyzed by an alanine scan (Wells, 1991) Meth. Enzymol. 202:390-411. In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore. Anti-CD163 antibodies are commercially available from Enzo Lifesciences and are suitable for use such assays.

Thus, one may design drugs which have, e.g., improved CD163 polypeptide activity or stability or which act as inhibitors, agonists, antagonists, etc. of CD163 polypeptide activity. By virtue of the availability of cloned CD163 sequences, sufficient amounts of the CD163 polypeptide may be made available to perform such analytical studies as x-ray crystallography. In addition, the knowledge of the CD163 protein sequence provided herein will guide those employing computer modeling techniques in place of, or in addition to x-ray crystallography.

In general, screening assays involve assaying the effect of a test agent on expression or activity of a target nucleic acid or target protein in a test sample (i.e., a sample containing the target nucleic acid or target protein). Expression or activity in the presence of the test compound or agent can be compared to expression or activity in a control sample (i.e., a sample containing the target protein that is incubated under the same conditions, but without the test compound). A change in the expression or activity of the target nucleic acid or target protein in the test sample compared to the control indicates that the test agent or compound modulates expression or activity of the target nucleic acid or target protein and is a candidate agent.

Compounds to be screened or identified using any of the methods described herein can include various chemical classes, though typically small organic molecules having a molecular weight in the range of 50 to 2,500 daltons. These compounds can comprise functional groups necessary for structural interaction with proteins (e.g., hydrogen bonding), and typically include at least an amine, carbonyl, hydroxyl, or carboxyl group, and preferably at least two of the functional chemical groups. These compounds often comprise cyclical carbon or heterocyclic structures and/or aromatic or polyaromatic structures (e.g., purine core) substituted with one or more of the above functional groups.

Compounds can be identified from a number of potential sources, including: chemical libraries, natural product libraries, and combinatorial libraries comprised of random peptides, oligonucleotides, or organic molecules. Chemical libraries consist of diverse chemical structures, some of which are analogs of known compounds or analogs or compounds that have been identified as “hits” or “leads” in other drug discovery screens, while others are derived from natural products, and still others arise from non-directed synthetic organic chemistry. Natural product libraries re collections of microorganisms, animals, plants, or marine organisms which are used to create mixtures for screening by: (1) fermentation and extraction of broths from soil, plant or marine microorganisms, or (2) extraction of plants or marine organisms. Natural product libraries include polypeptides, non-ribosomal peptides, and variants (non-naturally occurring) thereof. For a review, see Science 282:63-68 (1998). Combinatorial libraries are composed or large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of test compounds through the use of the various libraries herein permits subsequent modification of the test compound “hit” or “lead” to optimize the capacity of the “hit” or “lead” to prevent or suppress HIV infection.

In one embodiment, assays are provided for screening candidate or test molecules that are substrates of a target protein or a biologically active portion thereof in a cell. In another embodiment, the assays are for screening candidate or test compounds that interfere with HIV infection.

In yet another embodiment, a cell-free assay is provided in which a target protein (e.g., CD163) or biologically active portion thereof is contacted with a test compound and the ability of the test compound to bind to the target protein or biologically active portion thereof is evaluated. In general, biologically active portions of target proteins to be used in assays described herein include fragments that participate in interactions with other molecules, e.g., fragments with high surface probability scores.

Cell-free assays involve preparing a reaction mixture of the target proteins and the test compound under conditions and for a time sufficient to allow the two components to interact and bind, thus forming a complex that can be removed and/or detected. The ability of a target protein to bind to a target molecule can be determined using real-time Biomolecular Interaction Analysis (BIA) (e.g., Sjolander et al., Anal. Chem., 63:2338-2345, 1991, and Szabo et al., Curr. Opin. Struct. Biol., 5:699-705, 1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In several of these assays, the target proteins or the test substance is anchored onto a solid phase. The target protein/test compound complexes anchored on the solid phase can be detected at the end of the reaction. Generally, the target proteins are anchored onto a solid surface, and the test compound (which is not anchored) can be labeled, either directly or indirectly, with detectable labels discussed herein. It may be desirable to immobilize either the target protein, an anti-target protein antibody, or its target molecule to facilitate separation of complexed from uncomplexed forms of one or both of the proteins, as well as to accommodate automation of the assay. Binding of a test compound to a target protein, or interaction of a target protein with a target molecule in the presence and absence of a test compound, can be accomplished in any vessel suitable for containing the reactants. Examples of such vessels include microliter plates, test tubes, and micro-centrifuge tubes. In one embodiment, a fusion protein can be provided that adds a domain that allows one or both of the proteins to be bound to a matrix. For example, glutathione-S-transferase/target protein fusion proteins or glutathione-S-transferase/target fusion proteins can be adsorbed onto glutathione Sepharose™ beads (Sigma Chemical, St. Louis, Mo.) or glutathione derivatized microtiter plates, which are then combined with the test compound or the test compound and either the non-adsorbed target protein. The mixture is then incubated under conditions conducive to complex formation (e.g., at physiological conditions for salt and pH). Following incubation, the beads or microtiter plate wells are washed to remove any unbound components, the matrix immobilized in the case of beads, and the complex determined either directly or indirectly, for example, as described above. Alternatively, the complexes can be dissociated from the matrix, and the level of target protein binding or activity determined using standard techniques.

### III Therapeutics

The CD163 inhibitory agents of the invention can be formulated in pharmaceutical compositions. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

The following materials and methods are provided to facilitate the practice of the present invention.

Reagents. The NK1R antagonist, aprepitant (Emend®), manufactured by Merck & Co. (Whitehouse Station, N.J.), was purchased through the Children's Hospital of Philadelphia Pharmacy and purified by chromatography. Substance P (SP) and N-Formyl-Met-Leu-Phe (fMLP) were purchased from Sigma (St. Louis, Mo.). Neurokinin A (NKA) and Neurokinin B (NKB) were purchased from Phoenix Pharmaceuticals Inc. (Byrkubganem Calif.).

Virus. The M-tropic HIV-1 R5 strain Bal was obtained from the NIH AIDS Reagent Program. The HIV-GFP-tagged reporter virus pSF162R3 Nef+, was made based on HIV-SF162 strain and was a gift from Dr. Amanda Brown (John Hopkins School of Medicine, Baltimore, Md.) (45).

Monocyte isolation. Monocytes were obtained from the Human Immunology Core Facility of The University of Pennsylvania School of Medicine (Philadelphia, Pa.). The monocytes were purified from apheresis material using RossetteSep kits from Stem Cell Technologies. The RosetteSep kit was used to isolates monocytes from whole blood by negative selection. Unwanted cells were targeted for removal with Tetrameric Antibody Complexes (TAC) recognizing CD2, CD3, CD8, CD19, CD56, CD66b, CD123 and glycophorin A. After centrifugation over Ficoll-Paque™ PLUS (Catalog #07957), the unwanted cells were pelleted. The purified monocytes were present as a highly enriched population at the interface between the plasma and the buoyant density medium. The monocytes were washed in DMEM and finally cultured in DMEM supplemented with 10% fetal bovine serum (FBS), and antibiotics at 37° C. in 5% CO₂.

Intracellular calcium measurements. Freshly isolated monocytes were washed in Hank's balanced salt solution (HBSS) containing 1 mM calcium chloride then loaded with Fluo-4 AM (2 μM) and Fura-red AM (2 μM). Intracellular calcium recordings were performed using the Accuri C6 Analyzer (BD Biosciences, San Jose, Calif.). Fluorescence intensities were recorded in FL1-A (Fluo-4 AM) and FL3-A (Fura-red AM) channels. Intracellular calcium measurements were quantified by calculating the ratio between FL1-A/FL3-A values as a derived parameter in FlowJo 7.6.5 for Windows (Treestar Inc., Ashland, Oreg.) as previously described (1).

CD163 measurement by flow cytometry. For time-course experiments, freshly isolated monocytes were stimulated with either IL-10 (20 ng/ml) or SP (10 μM) and after 0, 2, 4 and 6 days of culture the cells were detached and the expression of membrane-bound CD163 was quantified by flow cytometry. For the dose-response experiments, freshly isolated monocytes were stimulated with either IL-10 (0.2, 0.6, 2, 6 or 20 ng/ml) or SP (0.3, 1, 3, 10 or 30 μM) and after 4 days of culture the cells were detached for CD163 measurement. Macrophages were detached using Lidocaine (4 mg/ml) in HBSS with EDTA (1 mM) for 30 minutes at 37° C. Cells were labeled with anti-human CD163 antibody (clone R-20) conjugated with PE (Trillium Diagnostics, Scarborough, Me.) for 30 minutes at room temperature. After labeling, cells were washed and resuspended in Hank's balanced salt solution (HBSS). Flow cytometry experiments were performed on an Accuri C6 analyzer (BD Biosciences, San Jose, Calif.) in the Flow Cytometry Core Laboratory of the Children's Hospital of Philadelphia Research Institute.

Cell Sorting. Macrophages cultured for 7 days were detached from a 6-well plate (1×106 cells/well) and labeled with anti-human CD163-PE for 30 minutes at room temperature. After labeling, cells were washed and resuspended in HBSS. Macrophages were sorted based on expression level of CD163 using a MoFlo XDP cell sorter (Beckman Coulter). The cells were plated in DMEM with 10% FBS and after 4 hours infected with HIV-Bal.

HIV infection. Macrophages were incubated with HIV-Bal virus overnight, and then extensively washed to remove unbound virus. The culture medium and the reagents were replaced twice weekly. At days 5, 7, 9 and 11 post HIV infection, cellular RNA was extracted from the MDM for assessment of HIV gag mRNA expression using real time RT-PCR assays.

RNA Extraction and Real-Time RT PCR Assays. Total RNA was extracted from macrophages using RNeasy kit (Qiagen), and the potential DNA contamination was eliminated by on-column DNase digestion as instructed by the manufacturer (Qiagen). RNA concentration was determined by Qubit fluorometer using Quant-iT RNA Assay kit (Invitrogen, Eugene, Oreg.).

Total RNA (1 μg) was reverse transcribed using AffinityScript QPCR cDNA Synthesis kit (STRATAGENE, Cedar Creek, Tex.) with random primer as instructed by the manufacture. Reverse transcriptase negative controls were used in order to control for genomic DNA contamination. 1 μl of the resulting cDNA was used as a template for real-time PCR amplification.

The sequences of the primers used are as follows: HIV gag Sense: CATGTTTTCAGCATTATCAGAAGGA (SEQ ID NO: 1), Antisense: TGCTTGATGTCCCCCCACT (SEQ ID NO:2), Probe: CACCCCACAAGATTTAAACACCATGCTAA (SEQ ID NO: 3); GAPDH Sense: GGTGGTCTCCTCTGACTTCAACA (SEQ ID NO: 4), Antisense: GTTGCTGTAGCCAAATTCGTTGT (SEQ ID NO: 5). All primers were synthesized by Integrated DNA Technologies, Inc. (Coralville, Iowa).

RNA Interference (RNAi). RNAi CD163 pool and control RNAi was purchased from Santa Cruz Biotechnologies. The siRNAs pool was transfected into macrophages with Lipofectamine RNAimax (Invitrogen), following the manufacturer's protocol. Experiments were carried out 48 h after transfection.

Microscopy. Macrophages were infected with HIV-GFP-tagged reported virus pSF162R3 Nef+. Infected cells were observed on a system including an Olympus IX51 microscope (Olympus America, Center Valley, Pa.), a Lambda LS 300W Xenon lamp, an external filter wheel and shutter, Lambda 10B controller (Sutter Instrument, Novato, Calif.), and Hamamatsu Orca C8484-03G02 digital camera (Hamamatsu Corp., Bridgewater, N.J.). GFP intensity was quantified using ImageJ (National Institute of Mental Health, Bethesda, Md., USA).

Statistical Analysis. The results are expressed as means±SE (n). Data were analyzed by Student's t-test for two group comparisons. Differences were considered significant when p<0.05.

The following example is provided to illustrate certain embodiments of the invention. It is not intended to limit the invention in any way.

### Example I

### Substance P Enhances HIV Infection in Macrophages Via the Haptoglobin-Hemoglobin Scavenger Receptor CD163

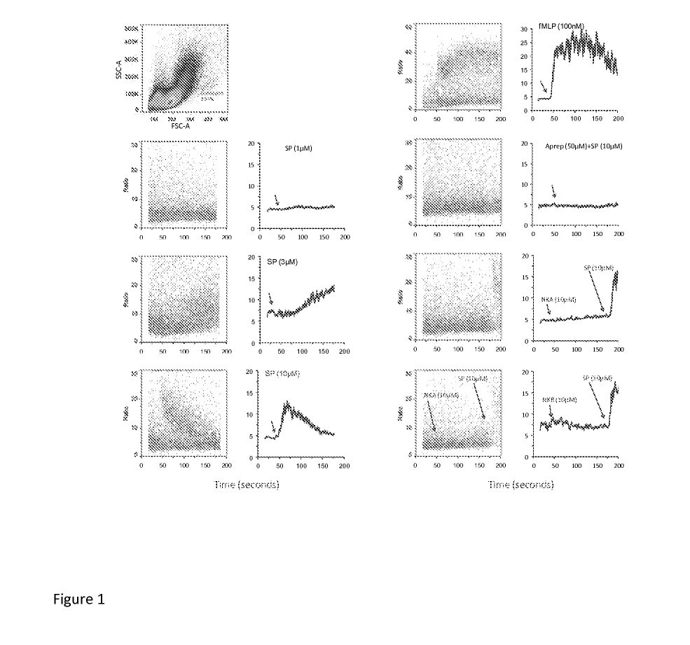

SP induces intracellular calcium increase in human monocytes. Our previous studies have shown that in U373MG cell NK1R stimulates the release of Ca²⁺ from intracellular stores though activation of phospholipase C. To assess whether the effect of SP on monocytes is due to activation of NK1R we examined the levels of intracellular Ca²⁺. We used a ratiometric calcium assay, where PBMC were loaded with Fluo-4 and Fura-red, then stimulated again with SP or appropriate agonist. Gating on lymphocyte and monocyte populations was performed based of light scattering properties of the cells (FIG. 1A). SP failed to induce any detectable change in intracellular Ca²⁺ in monocytes at concentrations of 1 μM or lower (FIG. 1B), whereas 3 or 10 μM SP induced intracellular Ca2+ increase (FIG. 1C, 1D). This pharmacology profile of NK1R, with high concentrations of SP required to elicit a response is consistent with the truncated form of NK1R. SP administered at concentrations as high as 30 μM did not induce intracellular Ca²⁺ increase in lymphocytes (data not shown).

We next confirmed that NK1R mediates the response to SP by pre-treating PBMC with the selective NK1R antagonist aprepitant (50 μM) for 15 minutes before adding SP. Aprepitant abolished SP induced calcium response (FIG. 1F). We also used fMLP as a positive control in order to ensure that the monocytes treated with Aprepitant were capable of a calcium response, as shown in FIG. 1B). Additionally, to ensure that the response to SP is not mediated by either NK2R or NK3R, we used the agonists NKA and NKB, respectively. NKA (10 μM) and NKB (10 μM) both failed to induce Ca²⁺ responses, while addition of SP induced a response in the same samples (FIGS. 1G and 1H).

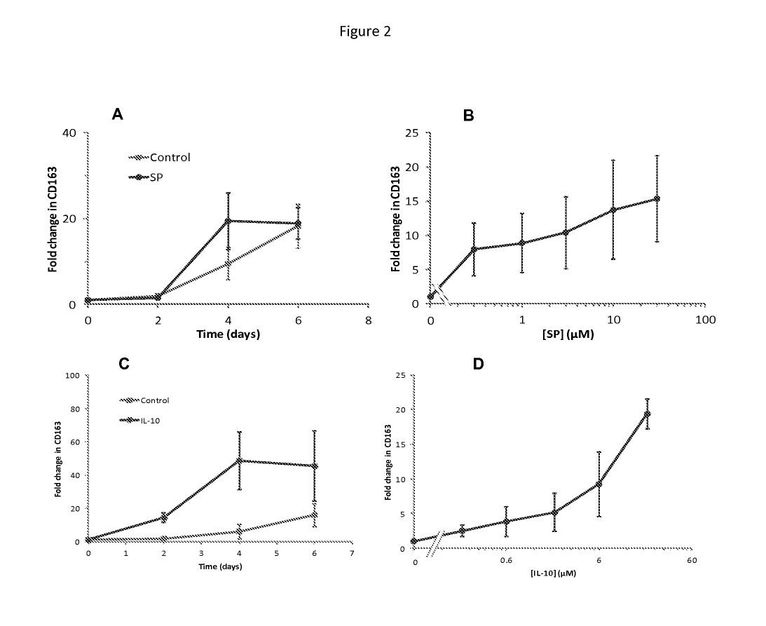

IL-10 increases expression of CD163 in human macrophages. We examined the expression of CD163 in response to IL-10. A time course of IL-10 stimulation of monocytes is shown in FIG. 2A). Notably, there is a significant increase in the expression of CD163 in as little as 2 days and this elevation is higher at days 4 and 6. Non treated cells also show an increase in CD163 over time, but to much lower levels as compared to cells treated with IL-10. A dose-response curve for the effect of IL-10 on CD163 expression as measured after 4 days of culture is shown in FIG. 2B). As little as 0.2 ng/ml of IL-10 induced a significant increase in CD163 expression, and treatment with 20 ng/ml of IL-10 had not induced maximal increase in CD163 expression.

SP increases expression of CD163 in human macrophages. We performed a time course study with 10 μM SP and we determined the dose-response relationship after 4 days stimulation with SP, in the same manner as described for IL-10. The time course of SP stimulation of monocytes is shown in FIG. 2C). SP induced a significant increase in CD163 in 4 days and this elevation did not persist at 6 days. FIG. 2D) shows the dose response for the effect of SP on CD163 expression as measured after 4 days. SP doses from 0.3-10 μM all showed significant increase in CD163 expression. Taken together our results indicate that SP induces expression of CD163, although at a lower extent than that induced by IL-10

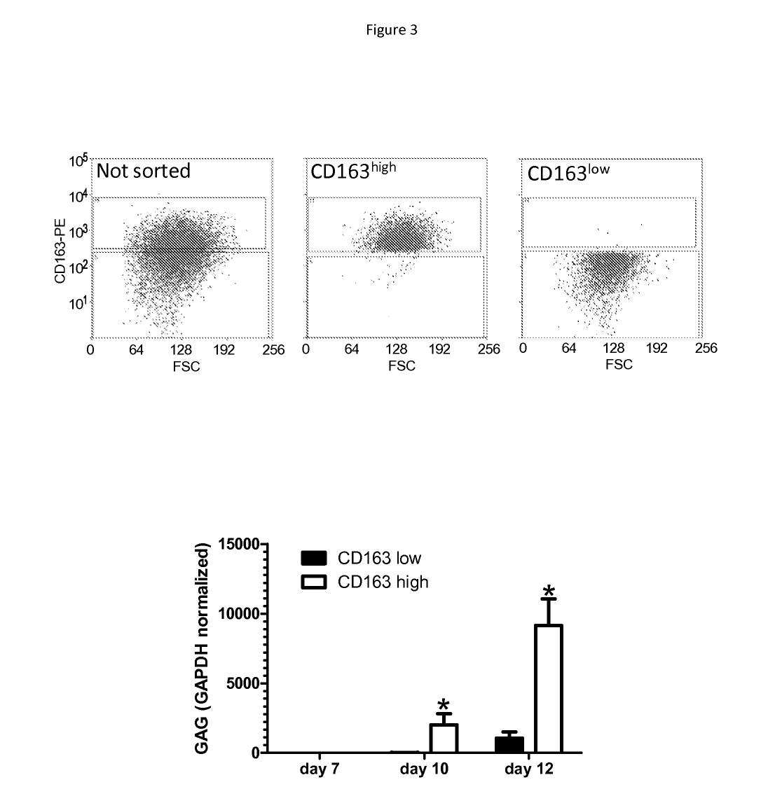

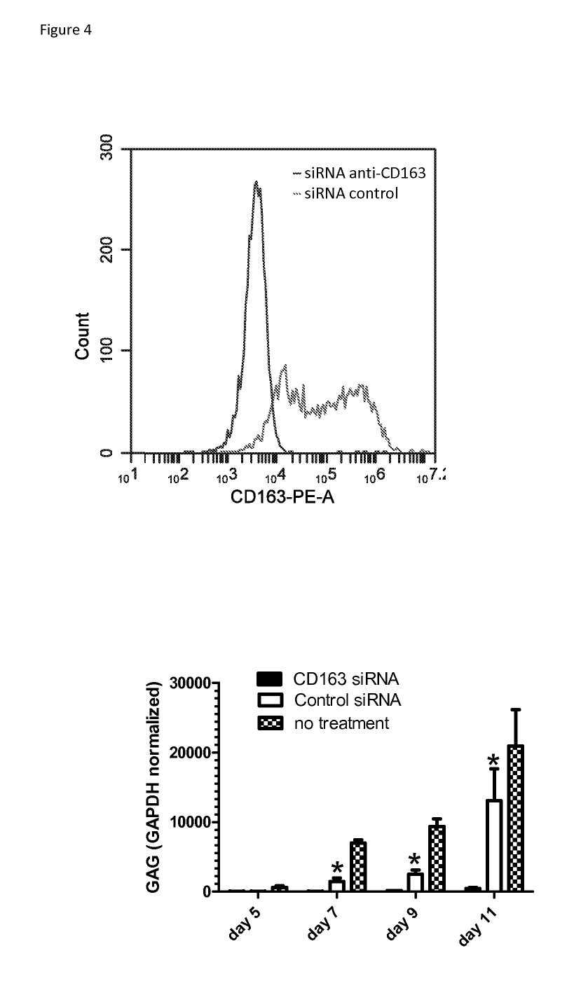

Macrophages with high levels of membrane-bound CD163 have increased susceptibility to HIV infection. To determine if susceptibility to HIV infection correlates with the levels of expression of CD163 on cell membrane, we stained macrophages with anti-CD163-PE antibody and performed cell sorting in two populations: CD163^{high} and CD163^{low}, (FIG. 3A) followed by infection of macrophages with HIV-Bal in 24 well plates and HIV infection was assayed by RT-PCR. We found that the productivity of infection CD163^{high} macrophages was much higher as compared to CD163^{low} cells (FIG. 3B). To bring additional evidence that high levels of membrane-bound CD163 promote HIV infection, we used siRNA to artificially decrease the expression of CD163 in MDM (FIG. 4A) and then we measured HIV infection by RT-PCR. We have found that the HIV infection of cells treated with siRNA targeting CD163 was almost completely inhibited, while control siRNA had a relatively modest, albeit statistically significant, inhibitory effect on macrophage infection.

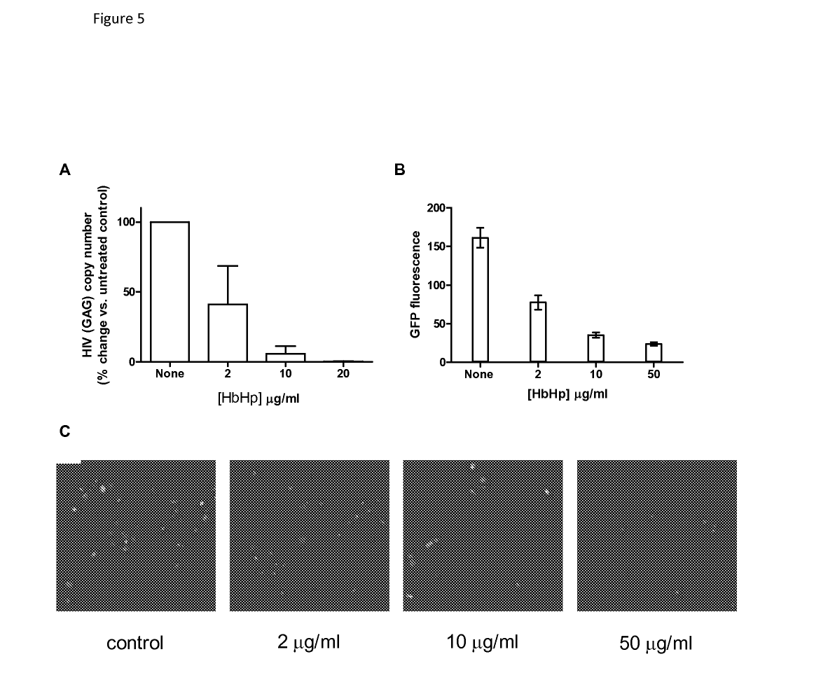

Hb-Hp complexes inhibit HIV infection in macrophages. Since CD163 is the prototypic scavenger receptor for Hb-Hp complexes, we determined if HIV infection is influenced by the presence of these complexes in culture media. For this set of experiments we used the HIV-GFP-tagged virus pSF162R3 Nef+(45), developed from the HIV-SF162 strain (FIGS. 5B and 5C) and also a non-tagged HIV-Bal virus (FIG. 5A). We found that Hb-Hp complexes caused a concentration-dependent decrease in number of infected cells/well (FIG. 5).

### Discussion

CD163 was initially described as a receptor responsible for binding and internalizing Hb-Hp complexes through endocytosis. More recently it has been found that CD163 is also a binding target for African swine fever virus (ASFV) (2) porcine reproductive and respiratory syndrome virus (PRRSV) (3). Although CD163 seems to effectively help with ASFV entry, it has been suggested that PPRSV and CD163 do not interact at the cell surface, but only intracellularly in early endosomes where CD163 facilitates PRRSV uncoating. CD163 can also mediate cytokine secretion in response to bacteria, or to initiate apoptosis via TNF-like weak inducer of apoptosis (TWEAK) (4).

It has been shown that SP enhances HIV infection in monocyte-derived macrophages and this effect may be mediated by an enhanced expression of the HIV co-receptor CCR5 (5,6) or by the activation of HIV LTR promoter (5). However, none of the mechanisms proposed until now completely explain the effect of SP, and it is more likely that multiple mechanisms are concurrently implicated in this phenomenon. In the present study we examined the effect of membrane-bound CD163 on HIV infection of macrophages, which contributes to the promoting effect of SP on HIV infection. This is consistent with the elevated levels of CD163 in HIV positive individuals (29-31). Our findings suggest that blocking SP stimulation or reducing the levels of CD163 expression may result in reducing overall HIV infection.

This is the first study to show that high concentrations of SP (10 μM) induce intracellular calcium mobilization in monocytes. Previous work had only shown that low doses of SP potentiated CCR5 mediated calcium mobilization (7). We determined that SP stimulation of monocytes leads to intracellular calcium increase through activation of NK1R. Previous work from our lab that showed that SP triggers intracellular Ca²⁺ increase in HEK293 cells transfected with the NK1R receptor (8) and also in U373MG cells which endogenously express the receptor (9), but SP (1 μM) failed to trigger Ca²⁺ increase in HEK293 cells expressing the truncated isoform of NK1R, consistent with the view that SP has much lower affinity for the truncated NK1R than for the full-length receptor. Thus, our findings strongly suggest that monocytes express a functional truncated NK1R which responds to high concentrations of SP.

Although plasma concentrations of SP are in the pM range, it is conceivable that in certain microenvironments (e.g. in the brain where SP is produced in large amounts in neurons) SP can reach much higher concentrations. To further demonstrate that NK1R mediates SP-induced calcium increase in monocytes, we showed that treatment with aprepitant, an NK1R selective antagonist, abolished the SP stimulated Ca²⁺ response. Although NK2R and NK3R can be activated by large concentrations of SP, we have shown that NK2R and NK3R agonists NKA and NKB did not elicit any change in Ca²⁺, giving further evidence that the effect of SP is mediated by NK1R receptor.

We found that the levels of membrane bound CD163 in monocytes cultured in AIM V medium and in the absence of FBS at 4 days after treatment with SP are progressively higher in cells treated with increasing doses of SP. This finding confirms that SP treatment leads to increased CD163 expression.

Freshly isolated monocytes express relatively high, homogenous levels of membrane-bound CD163. However, we found that the levels of CD163 vary in mature macrophages cultured under standard conditions, in the presence of FBS. In the experiments on sorted CD163^{high} and CD163^{low} macrophages we found that CD163 high monocytes were expressing levels of HIV-GAG significantly higher after 10 and 12 days post infection with HIV, suggesting that the presence of high levels of CD163 on macrophages facilitates HIV infection.

Simply classifying the monocytes into two groups of high and low expression of CD163 and seeing a difference in HIV infectivity does not prove that CD163 is the cause of the difference. To clearly demonstrate the hypothesis that membrane-bound CD163 plays a role in facilitating HIV infection, we knocked down the expression of CD163 using siRNA. The finding that the infection of cells treated with siRNA targeting CD163 was significantly lower as compared to cells treated with control siRNA strongly supports our hypothesis. Thus, the fact that SP induces enhanced HIV infection of macrophages can be explained by enhanced CD163 expression.

We have also found that in the presence of Hb-Hp complexes HIV infection is significantly attenuated. There are several explanations for this finding. It is possible that CD163 facilitates HIV entry, and the binding of HIV to the CD163 molecule is competitively inhibited in the presence of Hb-Hp complexes. However, we cannot rule out an alternative mechanism which can explain our finding based on the intracellular signaling capability of CD163. For example, it is also possible that during the internalization of Hb-Hp bound to CD163, some of the molecules that are critical for HIV entry, such as CCR5 or CD4, are also internalized, thus rendering cells less susceptible to HIV infection. Finally, it is also possible that Hb-Hp complexes bound to CD163 hinder the binding of HIV to CD4 and CCR5, thus inhibiting viral entry.

In conclusion, our study brings solid evidence that SP enhances the expression of membrane-bound CD163 in monocytes, thus shifting the differentiation of this cell type toward a macrophage phenotype that is more susceptible to HIV infection. We clearly show using multiple approaches that high levels of CD163 facilitate HIV infection of macrophages. Thus, it is conceivable this mechanism is responsible at least in part for the facilitating effect of SP on HIV infection.

The current study raises awareness on a possible role of CD163 in the maintenance of HIV reservoirs in CNS, where HIV infected CD163 positive macrophages may be exposed to high concentrations of SP. Targeting CD163 for the development of novel HIV therapies aimed at depleting HIV reservoirs in the brain and for HIV-associated neuro-cognitive disorders (HAND) may become a novel therapeutic strategy in the future.

### REFERENCES

1. Salthun-Lassalle, B., Traver, S., Hirsch, E. C., and Michel, P. P. (2005) Molecular pharmacology 68, 1214-1224
2. Severini, C., Improta, G., Falconieri-Erspamer, G., Salvadori, S., and Erspamer, V. (2002) Pharmacol Rev 54, 285-322
3. Pennefather, J. N., Lecci, A., Candenas, M. L., Patak, E., Pinto, F. M., and Maggi, C. A. (2004) Life Sci 74, 1445-1463
4. Page, N. M. (2005) Peptides 26, 1356-1368
5. Page, N. M. (2006) Vascul Pharmacol 45, 200-208
6. Werge, T. (2007) J Mol Recognit 20, 145-153
7. Lai, J. P., Ho, W. Z., Kilpatrick, L. E., Wang, X., Tuluc, F., Korchak, H. M., and Douglas, S. D. (2006) Proceedings of the National Academy of Sciences of the United States of America 103, 7771-7776
8. Lai, J. P., Lai, S., Tuluc, F., Tansky, M. F., Kilpatrick, L. E., Leeman, S. E., and Douglas, S. D. (2008) Proceedings of the National Academy of Sciences of the United States of America 105, 12605-12610
9. Fong, T. M., Anderson, S. A., Yu, H., Huang, R. R., and Strader, C. D. (1992) Molecular pharmacology 41, 24-30
10. Douglas, S. D., Ho, W. Z., Gettes, D. R., Cnaan, A., Zhao, H., Leserman, J., Petitto, J. M., Golden, R. N., and Evans, D. L. (2001) AIDS (London, England) 15, 2043-2045
11. Ho, W. Z., and Douglas, S. D. (2004) Journal of neuroimmunology 157, 48-55
12. Lai, J. P., Ho, W. Z., Zhan, G. X., Yi, Y., Collman, R. G., and Douglas, S. D. (2001) Proceedings of the National Academy of Sciences of the United States of America 98, 3970-3975
13. Wang, X., Douglas, S. D., Lai, J. P., Tuluc, F., Tebas, P., and Ho, W. Z. (2007) J Neuroimmune Pharmacol 2, 42-48
14. Douglas, S. D., Cnaan, A., Lynch, K. G., Benton, T., Zhao, H., Gettes, D. R., and Evans, D. L. (2008) AIDS research and human retroviruses 24, 375-378
15. Douglas, S. D., and Leeman, S. E. (2011) Annals of the New York Academy of Sciences 1217, 83-95
16. Manak, M. M., Moshkoff, D. A., Nguyen, L. T., Meshki, J., Tebas, P., Tuluc, F., and Douglas, S. D. (2010) AIDS (London, England) 24, 2789-2796
17. Burdo, T. H., Lentz, M. R., Autissier, P., Krishnan, A., Halpern, E., Letendre, S., Rosenberg, E. S., Ellis, R. J., and Williams, K. C. (2011) The Journal of infectious diseases 204, 154-163
18. Tippett, E., Cheng, W. J., Westhorpe, C., Cameron, P. U., Brew, B. J., Lewin, S. R., Jaworowski, A., and Crowe, S. M. (2011) PloS one 6, e19968
19. Hearps, A. C., Maisa, A., Cheng, W. J., Angelovich, T. A., Lichtfuss, G. F., Palmer, C. S., Landay, A. L., Jaworowski, A., and Crowe, S. M. (2012) AIDS (London, England) 26, 843-853
20. Burdo, T. H., Lo, J., Abbara, S., Wei, J., DeLelys, M. E., Preffer, F., Rosenberg, E. S., Williams, K. C., and Grinspoon, S. (2011) The Journal of infectious diseases 204, 1227-1236
21. Subramanian, S., Tawakol, A., Burdo, T. H., Abbara, S., Wei, J., Vijayakumar, J., Corsini, E., Abdelbaky, A., Zanni, M. V., Hoffmann, U., Williams, K. C., Lo, J., and Grinspoon, S. K. (2012) JAMA 308, 379-386
22. Knudsen, T. B., Gustafson, P., Kronborg, G., Kristiansen, T. B., Moestrup, S. K., Nielsen, J. O., Gomes, V., Aaby, P., Lisse, I., Moller, H. J., and Eugen-Olsen, J. (2005) Clin Microbiol Infect 11, 730-735
23. Graversen, J. H., Madsen, M., and Moestrup, S. K. (2002) The international journal of biochemistry & cell biology 34, 309-314
24. Kristiansen, M., Graversen, J. H., Jacobsen, C., Sonne, O., Hoffman, H. J., Law, S. K., and Moestrup, S. K. (2001) Nature 409, 198-201
25. Moller, H. J., Peterslund, N. A., Graversen, J. H., and Moestrup, S. K. (2002) Blood 99, 378-380
26. Moreno, J. A., Ortega-Gomez, A., Delbosc, S., Beaufort, N., Sorbets, E., Louedec, L., Esposito-Farese, M., Tubach, F., Nicoletti, A., Steg, P. G., Michel, J. B., Feldman, L., and Meilhac, O. (2012) European heart journal 33, 252-263
27. Hogger, P., Dreier, J., Droste, A., Buck, F., and Sorg, C. (1998) J Immunol 161, 1883-1890
28. Buechler, C., Ritter, M., Orso, E., Langmann, T., Klucken, J., and Schmitz, G. (2000) Journal of leukocyte biology 67, 97-103
29. Fischer-Smith, T., Tedaldi, E. M., and Rappaport, J. (2008) AIDS research and human retroviruses 24, 417-421
30. Kim, W. K., Alvarez, X., Fisher, J., Bronfin, B., Westmoreland, S., McLaurin, J., and Williams, K. (2006) The American journal of pathology 168, 822-834
31. Soulas, C., Conerly, C., Kim, W. K., Burdo, T. H., Alvarez, X., Lackner, A. A., and Williams, K. C. (2011) The American journal of pathology 178, 2121-2135
32. Fischer-Smith, T., Bell, C., Croul, S., Lewis, M., and Rappaport, J. (2008) Journal of neurovirology 14, 318-326
33. Moller, H. J. (2012) Scandinavian journal of clinical and laboratory investigation 72, 1-13
34. Etzerodt, A., Maniecki, M. B., Moller, K., Moller, H. J., and Moestrup, S. K. (2010) Journal of leukocyte biology 88, 1201-1205
35. Ragin, A. B., Wu, Y., Ochs, R., Du, H., Epstein, L. G., Conant, K., and McArthur, J. C. (2011) Journal of neurovirology 17, 153-158
36. Fabriek, B. O., Moller, H. J., Vloet, R. P., van Winsen, L. M., Hanemaaijer, R., Teunissen, C. E., Uitdehaag, B. M., van den Berg, T. K., and Dijkstra, C. D. (2007) Journal of neuroimmunology 187, 179-186
37. Russell, R. E., Culpitt, S. V., DeMatos, C., Donnelly, L., Smith, M., Wiggins, J., and Barnes, P. J. (2002) Am J Respir Cell Mol Biol 26, 602-609
38. Shapiro, S. D., Kobayashi, D. K., and Ley, T. J. (1993) The Journal of biological chemistry 268, 23824-23829
39. Davis, B. H., and Zarev, P. V. (2005) Cytometry 63, 16-22
40. Kneidl, J., Loffler, B., Erat, M. C., Kalinka, J., Peters, G., Roth, J., and Barczyk, K. (2012) Cell Microbiol 14, 914-936
41. Timmermann, M., Buck, F., Sorg, C., and Hogger, P. (2004) Immunol Cell Biol 82, 479-487
42. Frings, W., Dreier, J., and Sorg, C. (2002) FEBS Lett 526, 93-96
43. Boyer, L. C., Cardo-Vila, M., Kuniyasu, A., Sun, J., Rangel, R., Takeya, M., Aggarwal, B. B., Arap, W., and Pasqualini, R. (2007) J Immunol 178, 8183-8194
44. Lichtner, M., Maranon, C., Vidalain, P. O., Azocar, O., Hanau, D., Lebon, P., Burgard, M., Rouzioux, C., Vullo, V., Yagita, H., Rabourdin-Combe, C., Servet, C., and Hosmalin, A. (2004) AIDS research and human retroviruses 20, 175-182
45. Brown, A. M. (2009) Methods Mol Biol 515, 165-175
46. Chen, P., Douglas, S. D., Meshki, J., and Tuluc, F. (2012) PloS one 7, e45322
47. Sanchez-Tones, C., Gomez-Puertas, P., Gomez-del-Moral, M., Alonso, F., Escribano, J. M., Ezquerra, A., and Dominguez, J. (2003) Arch Virol 148, 2307-2323
48. Van Gorp, H., Van Breedam, W., Van Doorsselaere, J., Delputte, P. L., and Nauwynck, H. J. (2010) J Virol 84, 3101-3105
49. Van Gorp, H., Delputte, P. L., and Nauwynck, H. J. (2010) Mol Immunol 47, 1650-1660
51. Chernova, I., Lai, J. P., Li, H., Schwartz, L., Tuluc, F., Korchak, H. M., Douglas, S. D., and Kilpatrick, L. E. (2009) Journal of leukocyte biology 85, 154-164
53. Meshki, J., Douglas, S. D., Hu, M., Leeman, S. E., and Tuluc, F. (2011) PloS one 6, e25332

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

1. A method for inhibiting HIV infection and or latency in a patient in need thereof, comprising administration of an effective amount of an agent which inhibits CD163 receptor activity, said agent being effective to impede HIV uptake into target cells.

2. The method of claim 1, wherein said agent is a small molecule, an inhibitory RNAi or an antibody immunologically specific for CD163 or a functional fragment thereof.

3. The method of claim 2, wherein said agent is an RNAi, said RNAi being effective to downmodulate expression of CD163.

4. A screening method for identifying agents useful for the treatment of HIV infection, comprising;
a) incubating a population of monocytes expressing CD163 in the presence and absence of said test agent,
b) contacting said monocytes with a compound that detectably labels said CD163, thereby determining CD163 expression levels in the presence or absence of said agent, agents which inhibit expression of CD163 in treated cells relative to untreated cells being useful for inhibiting HIV infection in said cell.

5. The method of claim 4, wherein said agent is an siRNA and said compound is a detectably labeled antibody.

6. The method of claim 4, wherein said agent comprises Hb-Hp complexes.

7. The method of claim 4, wherein said agent inhibits SP induced expression of CD163.

8. The method of claim 4, wherein said cell is a macrophage.
